(19) United States
(12) Patent Application Publication
(10) Pub. No.: US 2019/0046350 A1
(43) Pub. Date: Feb. 14, 2019

(54) SYSTEM FOR DELIVERING HEAT THERAPY VIA THERMALLY CONTROLLED AIR

(71) Applicant: 3M INNOVATIVE PROPERTIES COMPANY, St. Paul, MN (US)

(72) Inventors: Thomas P. Anderson, Golden Valley, MN (US); Diane L. Emslander, Stillwater, MN (US); Dominic J. Julian, Woodbury, MN (US); Michelle L. Legatt, Ventura, CA (US); Kristin M. Luke, Shoreview, MN (US); David J. Prince, St. Paul, MN (US); Tanushree Thote, Sacramento, CA (US)

(21) Appl. No.: 16/076,846

(22) PCT Filed: Feb. 8, 2017
(86) PCT No.: PCT/US2017/016943

§ 371 Date: Aug. 9, 2018

Related U.S. Application Data
(60) Provisional application No. 62/294,168, filed on Feb. 11, 2016.

(51) Int. Cl. A61F 7/00; A61F 7/02; A61F 7/08

(52) U.S. Cl. CPC A61F 7/0085; A61F 7/02; A61F 7/08; A61F 2007/0032; A61F 2007/004; A61F 2007/0228; A61F 2007/0094; A61F 2007/0042; A61F 2007/0091; A61F 2007/0055; A61F 2007/0093

(57) ABSTRACT

The present disclosure describes a system for providing targeted, temperature regulated therapy to a user. The system includes a convective unit and a forced air controller, with the convective unit including a sleeve for securing to a desired portion of the user's anatomy. The convective unit is fluidly coupled to the forced air controller, which is operable to transport a thermally conditioned air stream to an inflatable chamber in the sleeve. The convective unit receives the air stream, inflates, distributes the typically warmed, pressurized air within the inflatable chamber, and emits the air through one or more air permeable surfaces for convective transfer of heat to the body of the wearer enveloped by the sleeve.

### BACKGROUND

Heat and cold are the two most common types of noninvasive and non-addictive pain-relief therapies for muscle and joint pain. In general, a new injury will cause inflammation and possibly swelling. Ice will decrease the blood flow to the injury, thereby decreasing inflammation and swelling. Pain that recurs can be treated with heat, which will bring blood to the area and promote healing.

Heat can serve to open up blood vessels, which increases blood flow and supplies oxygen and nutrients to reduce pain in an effected area (e.g., muscles, ligaments, and tendons, particular those proximate a joint). The warmth from the heat can also serve to decrease muscle spasms and can increase range of motion, particularly at a joint. Applying superficial heat to a wearer's body can improve the flexibility of tendons and ligaments, reduce muscle spasms, and alleviate pain. The heat is typically maintained at a consistent temperature and applied to area for only 15-20 minutes at a time.

### SUMMARY OF THE INVENTION

The present disclosure provides depicts a system for providing targeted, temperature regulated therapy to a user. The system includes a convective unit and a forced air controller, with the convective unit including a sleeve for securing to a desired portion of the user's anatomy. The convective unit is fluidly coupled to the forced air controller, which is operable to transport a thermally conditioned air stream to an inflatable chamber in the sleeve. The convective unit receives the air stream, inflates, distributes the typically warmed, pressurized air within the inflatable chamber, and emits the air through one or more air permeable surfaces for convective transfer of heat to the body of the wearer enveloped by the sleeve. The systems described can provide the desired heat for the 15-20 minute recommended time and then blow ambient air to cool the area down. This would provide controlled heat therapy without any thoughts or work from the user, as is necessary for typical heat pads or similar therapeutic solutions.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

In this application, terms such as “a”, “an”, and “the” are not intended to refer to only a singular entity, but include the general class of which a specific example may be used for illustration. The terms “a”, “an”, and “the” are used interchangeably with the term “at least one.” The phrases “at least one of” and “comprises at least one of” followed by a list refers to any one of the items in the list and any combination of two or more items in the list.

As used herein, the term “or” is generally employed in its usual sense including “and/or” unless the content clearly dictates otherwise.

The term “and/or” means one or all of the listed elements or a combination of any two or more of the listed elements.

Also herein, all numbers are assumed to be modified by the term “about” and preferably by the term “exactly.” As used herein in connection with a measured quantity, the term “about” refers to that variation in the measured quantity as would be expected by the skilled artisan making the measurement and exercising a level of care commensurate with the objective of the measurement and the precision of the measuring equipment used.

Also herein, the recitations of numerical ranges by endpoints include all numbers subsumed within that range as well as the endpoints (e.g., 1 to 5 includes 1, 1.5, 2, 2.75, 3, 3.80, 4, 5, etc.).

As used herein as a modifier to a property or attribute, the term “generally”, unless otherwise specifically defined, means that the property or attribute would be readily recognizable by a person of ordinary skill but without requiring absolute precision or a perfect match (e.g., within +/−20% for quantifiable properties). The term “substantially”, unless otherwise specifically defined, means to a high degree of approximation (e.g., within +/−10% for quantifiable properties) but again without requiring absolute precision or a perfect match. Terms such as same, equal, uniform, constant, strictly, and the like, are understood to be within the usual tolerances or measuring error applicable to the particular circumstance rather than requiring absolute precision or a perfect match.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

FIG. 1 is an illustration of a forced air temperature regulation system according to one embodiment of the present disclosure;

FIG. 2 is an illustration of a forced air temperature regulation system according to another embodiment of the present disclosure;

FIG. 3 is an illustration of a forced air temperature regulation system according to another embodiment of the present disclosure;

FIG. 4 is an illustration of a forced air temperature regulation system according to another embodiment of the present disclosure; and

FIG. 5 is an illustration of a forced air temperature regulation system according to another embodiment of the present disclosure.

While the above-identified figures set forth several embodiments of the disclosure other embodiments are also contemplated, as noted in the description. In all cases, this disclosure presents the invention by way of representation and not limitation. It should be understood that numerous other modifications and embodiments can be devised by those skilled in the art, which fall within the scope and spirit of the principles of the invention.

### DETAILED DESCRIPTION OF ILLUSTRATIVE EMBODIMENTS

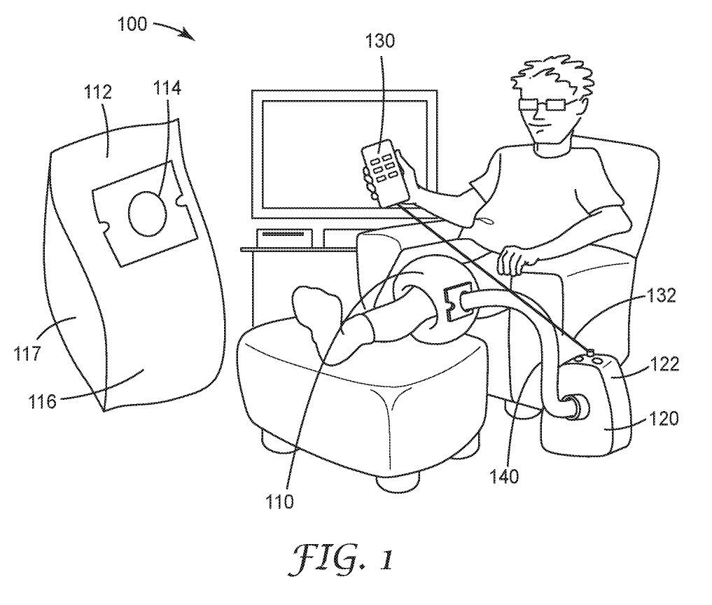

FIG. 1 depicts a system 10 for providing temperature regulated therapy to a user. The system includes a convective unit 110, a forced air controller 120, and a remote 130. The convective unit 110 includes a sleeve 112 for securing to a desired portion of the anatomy and is fluidly coupled to the forced air controller via flexible conduit 140. The conduit 140 serves to transport thermally conditioned air from the forced air controller 120 to an inflatable chamber (not shown) in the sleeve 112.

While the convective unit 110 is shown wrapped around a wearer's knee, those of skill in the art will appreciate that convective units may also be constructed for enveloping ankles, elbows, knees, and hip, for example. In certain embodiments, the shape, contour and construction of the convective unit will depend on the specific joint covered. In other embodiments, the convective unit 10 may be provided with fasteners and other mechanisms for adjusting the dimensions of the sleeve to accommodate a variety of different portions of the wearer's anatomy. Typically, the sleeve will circumferentially surround, either wholly or in part, the particular joint needing therapy.

The sleeve 112 defines a passage for receipt of an area to be treat and further includes an inflatable chamber (not shown), such as a bladder, disposed within at least a portion of the sleeve body. As used herein, “inflatable” refers to a structure which increases in volume when air or other gas is supplied at a pressure greater than atmospheric pressure to the interior of the structure. The inflatable chamber typically includes at least one inflation port constructed to receive and retain the end of a conduit configured to deliver pressurized air into the chamber. In FIG. 1, access to the interior chamber is provided by inflation port 114, in which a nozzle or connector of the fluid conduit 140 is retained.

The sleeve 112 includes a skin facing surface 118 designed, as implied, to be disposed on the wearer's skin or clothing when the system 100 is employed. The skin facing surface 118 preferably includes an air permeable material, so as to allow the heat from thermally controlled air in the interior chamber to convectively transfer to the body of the wearer. Suitable air permeable materials include, for example, woven fabrics, nonwoven fabrics, perforated film (e.g., film including slits, apertures, and interstices), porous film, laminated material (e.g., nonwoven fabrics with perforated film, etc.), flocked fabrics, and the like. Nonwoven fabrics include, for example, carded thermally bonded nonwovens, spunbond nonwovens, hydroentangled/spunlaced nonwovens, SMS (Spunbond-Meltblown-Spunbond) nonwovens, airlaid nonwovens, wet-laid nonwovens, or the like. In some or all embodiments, the outer surface 116 of the sleeve may incorporate one or more air impermeable materials (i.e., material having less air permeability than the material at skin facing surface 118). Air impermeable materials include, for example, single layer plastic film (e.g., Polyethylene, Propylene, Polyurethane, polyester, etc.), metal film (e.g., aluminum foil film, etc.), elastic film (e.g., polyurethane, etc.), multi-layer film (e.g., co-extruded film, blown film, etc.), film coated paper, and the like.

Illustrative examples of convective unit (i.e., convective devices) are described in U.S. Pat. Nos. 6,876,884, 7,014,431 7,276,076, 7,520,889, 7,749,261, and 7,871,428.

In operation, the forced air controller 120 produces a stream of pressurized, heated air which exits the controller housing 122 at some air stream temperature (which may range, for example, from ambient to an elevated level) and some air stream velocity into the one end of the conduit 140. This air stream is conducted by the conduit 140 and into the convective unit 110 through the inflation port 114. The convective unit 110 receives the air stream, inflates, distributes the typically warmed, pressurized air within the inflatable structure, and emits the air through one or more air permeable surfaces for convective transfer of heat to the body of the wearer. Illustrative examples of force air controller construction and operation are described in U.S. Pat. Nos. 6,876,884; 7,819,911; and 7,976,572.

The forced air controller 120 can include a user interface (not shown) that allows a user to manually control the temperature of the system, the duration of treatment and other aspects of the system. The force air controller 120 can include a processor configured to cycle between distributing warm, pressurized air and returning the temperature of the air to room temperature. Functions described herein can be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the requisite function. A general purpose processor can be a microprocessor, but in the alternative, the processor can be any conventional processor, controller, microcontroller, or state machine. A processor can also be implemented as a combination of computing devices, e.g., a combination of a DSP and a microprocessor, a plurality of microprocessors, one or more microprocessors in conjunction with a DSP core, or any other such configuration.

In some or all implementations (including the embodiment depicted in FIG. 1), a manually-operated remote control 130 may be connected by signal cable 134 to control circuitry (not shown) disposed within the controller housing 122. In presently preferred embodiments, the remote control 130 enables the person wearing the convective unit 110 to regulate at least one of the temperature, pressure, and velocity of the stream of air produced by the forced air controller 120 and thereby to control factors affecting the thermal comfort of his or her anatomy underlying sleeve 112. The remote 130 can (in addition to or in lieu of the user interface on controller 120) include a user interface for presenting information to the user. The user interface may include a display and/or a speaker. The user interface may include one or more input devices and/or output devices so that the user can communicate with force air controller 120. User interface may include a touch-sensitive and/or a presence-sensitive screen, a voice responsive system, or any other type of device for detecting a command from a user. In one example, the user interface may be a touch screen interface. In other examples, the user interface may include a display and one or more buttons, pads, joysticks, mice, tactile device, or any other device capable of turning user actions into electrical signals that control forced air controller 120.

The remote control 130 can include at least two controls, one (temperature control) for control of the thermal condition of pressurized, heated air produced by the forced air controller 120, the other for regulating high voltage functions of the unit (i.e., allowing for the user to turn the forced air controller on and off). The remote control 130 can also, in some or all implementations, include a separate control for regulating the pressure of the heated air within the inflatable chamber.

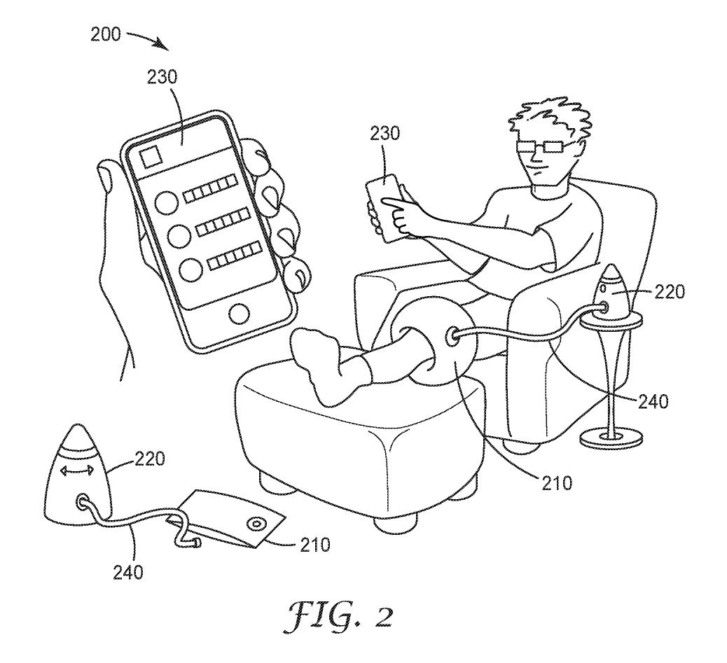

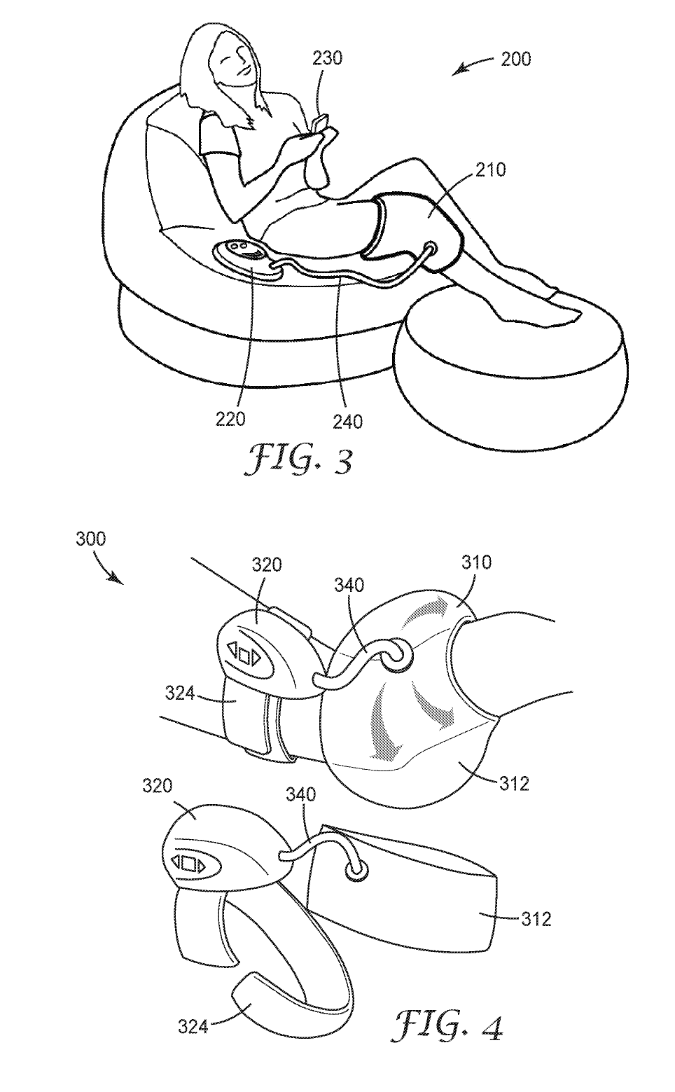

FIGS. 2 and 3 depict another implementation of a user controlled system 200 for providing temperature regulated therapy to a user. Similar to system 100, the user controlled system 200 includes a convective unit 210, a forced air controller 220, a remote 230, and a conduit 240. Those skilled in the art will perceive that certain functional elements of convective unit 110, forced air controller 120, remote 130, and a conduit 140 apply mutatis mutandis to convective unit 210, forced air controller 220, remote 230, and conduit 240, and need not be repeated at length here. Unlike remote 130, the remote 230 is not electrically or functionally coupled to forced air controller 220 via a signal cable. Remote control 230 is configure to wirelessly communicate with forced air controller 220. The remote control 230 may include a telemetry module utilizing any short-range communication (e.g., Bluetooth or Near-Field Communication) or other public or proprietary wireless communication protocols. For example, in some embodiments, the telemetry module relies on a Class 1 or Class 2 Bluetooth radio. In presently preferred implementations, the remote 230 is a mobile computing device (e.g., “smart phone”). The mobile computing device may include one or more processors, microprocessors, internal memory and/or data storage and other electronic circuitry for executing software or firmware to provide the functionality described herein.

FIG. 4 depicts another implementation of a therapeutic heating system 300 according to the present disclosure. Like therapeutic heating systems 100 and 200, the heating system 300 includes a convective unit 310 and a force air controller 320. Those skilled in the art will perceive that certain functional elements of convective unit 110, forced air controller 120, and conduit 140 apply mutatis mutandis to convective unit 310, forced air controller 320, and conduit 340, and need not be repeated at length here. In contrast to the embodiments depicted in FIGS. 1-3, the force air controller 320 is designed to be worn by the user. The forced air controller 320 can be mounted on or otherwise coupled to an adjustable strap 324 that is releasably engagable with a wearer's appendage. The strap 324 typically comprises a flexible belt or like ribbon that can be wrapped around the appendage and secured. The strap typically comprises a relatively inelastic material (for example, a material having no more than about 30% stretch under tension) such as foam laminates or a woven cotton or nylon. The strap 324 may also comprise an engaging surface similar, for example, to loop in a “hook and loop” application applied to either or both sides of the strap. The width of the strap can help to distribute the applied circumferential force around the wearer's appendage so the forced air control unit 320 is held on firmly but still comfortable.

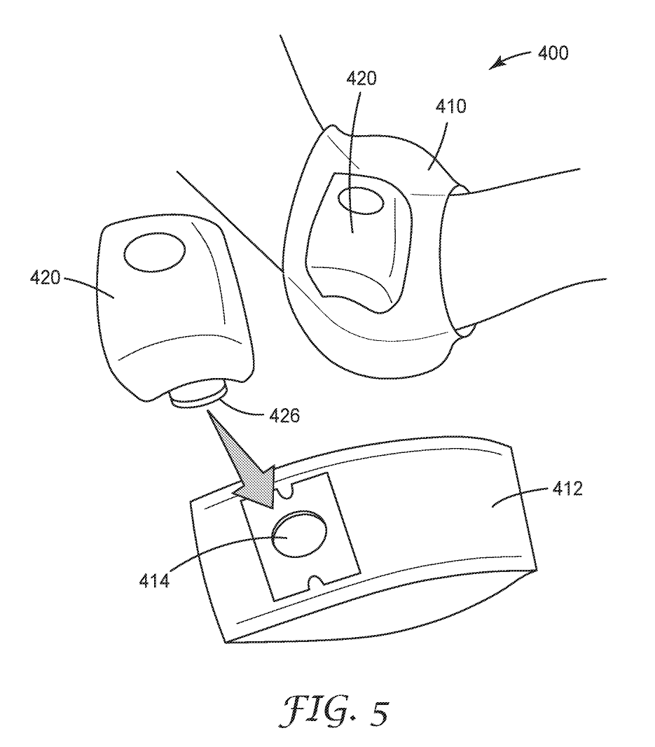

FIG. 5 depicts a wearable therapeutic heating system 400 featuring a force air controller 420 directly coupled to the convective unit 410. The force air controller 420 includes a nozzle 426 for coupling directly with inflation port 414 on sleeve 412. This configuration obviates the need for a separate remote or conduit connection.

All of the patents and patent applications mentioned above are hereby expressly incorporated by reference. The embodiments described above are illustrative of the present invention and other constructions are also possible. Accordingly, the present invention should not be deemed limited to the embodiments described in detail above and shown in the accompanying drawings, but instead only by a fair scope of the claims that follow along with their equivalents.

1. A system for selectively heating a portion of a wearer's anatomy, the system comprising
a convective unit including a sleeve for securing around the portion of the wearer's anatomy;
a forced air controller fluidly coupled to the sleeve for directing thermally controlled air into the sleeve; and
a remote control having a user interface that allows the wearer to control the temperature of the thermally controlled air;
wherein the remote control is configured to wirelessly communicate with the forced air controller.

2. The system of claim 1, wherein the sleeve further comprises:
a skin-facing surface capable of being disposed adjacent to the wearer's skin or clothing when the system is in use, the skin-facing surface including an air permeable material; and
an outer surface of the sleeve including an air impermeable material.

3. The system of claim 2, wherein the air permeable material is at least one of a woven fabric, a nonwoven fabric, a perforated film, a porous film, a laminated material, a flocked fabric, or a combination thereof.

4. The system of claim 3, wherein the nonwoven fabric includes at least one of a carded thermally bonded nonwoven, a spunbond nonwoven, a hydroentangled/spunlaced nonwoven, a spunbond-meltblown-spunbond nonwoven, an airlaid nonwoven, a wet-laid nonwoven, or a combination thereof.

5. The system of claim 2, wherein the impermeable material includes at least one of a single layer plastic film, a metal film, an elastic film, a multi-layer film, a film coated paper, or a combination thereof.

6. The system of claim 1, further comprising:
one or more fasteners on the sleeve and/or convective unit.

7. The system of claim 1, wherein the forced air controller includes a processor configured to cycle between distributing warm, pressurized air and room temperature air.

8. The system of claim 1, wherein the remote control wirelessly communicates via a telemetry module utilizing short-range communication or other public or proprietary wireless communication protocols.

9. The system of claim 1, wherein the remote control is a mobile computing device.

10. The system of claim 9, wherein the mobile computing device is a smart phone.
